(19) United States
(12) Patent Application Publication
(10) Pub. No.: US 2011/0319831 A1
(43) Pub. Date: Dec. 29, 2011

(54) PROTECTION ASSEMBLY

(75) Inventor: Andreas Bode, Frankfurt am Main (DE)

(73) Assignee: SANOFI-AVENTIS DEUTSCHLAND GMBH, Frankfurt am Main (DE)

(21) Appl. No.: 13/131,519

(22) PCT Filed: Dec. 8, 2009
(86) PCT No.: PCT/EP2009/066571

§ 371 Date: Sep. 15, 2011

(30) Foreign Application Priority Data
Dec. 12, 2008 (EP) 08021568.4

(51) Int. Cl. A61M 5/32

(52) U.S. Cl. 604/192

(57) ABSTRACT

A protection assembly is disclosed that combines a cover to preserve sterility of a sharp instrument with a trap to prevent accidental sticks where in one embodiment the cover is detached from the protection assembly and disposed and in another embodiment the cover is permanently attached to the protection assembly.

### CROSS REFERENCE TO RELATED APPLICATIONS

The present application is a U.S. National Phase Application pursuant to 35 U.S.C. §371 of International Application No. PCT/EP2009/066571 filed Dec. 8, 2009, which claims priority to EP Patent Application No. 08021568.4 filed on Dec. 12, 2008. The entire disclosure content of these applications are herewith incorporated by reference into the present application.

### TECHNICAL FIELD

The invention relates to a combination of a protective cover and a protective trap mechanism to prevent accidental sticking by sharp instruments. Specifically, the invention is useful for needles attached to medication delivery devices, e.g. single-use disposable syringes.

### BACKGROUND

The risks associated with the spread of blood-borne pathogens through use of sharp instruments, such as hypodermic needles and scalpels, are significant and well documented. In recent years, the public has become increasingly aware of the health hazards associated with needle reuse and accidental needle prickings. These risks are most prevalent among certain groups of people, such as drug addicts, drug users (e.g., diabetics), medical personnel and healthcare providers. In fact, more than twenty blood-borne pathogens can be transmitted by the reuse of needles or accidental needle prickings, just a few of which include human immunodeficiency virus (HIV), acquired immunodeficiency syndrome (AIDS), hepatitis B, hepatitis C, syphilis, malaria, tuberculosis, and herpes.

To avoid such injuries, it is desirable to equip sharp medical devices with protective mechanisms that offer a protection after the sharp instrument is used. However, it is equally important and desirable to maintain complete sterility of the sharp instrument before use as well as to prevent accidental sticks.

The art has long recognized the use of a protective cap or cover that completely surrounds the sharp instrument and that maintains sterility up until the time of use. However, these caps are made to be completely removed from the device, and in most cases, discarded before the device is used. Likewise, the art has taught the use of shrink wrap films that can surround a device and are then used to trap the sharp instrument after it is used. Such a system is disclosed in CA 2 603 841. However, such systems provide no means to maintain the sterility of the sharp instrument and typically must be used in conjunction with a separate traditional protective cap as described above.

### SUMMARY

The invention solves the above-mentioned problems by providing a unitary protection assembly for an instrument having sharp means. The protection assembly comprises a cover configured to cover the sharp means and protection means having a mount for attaching the protection means to the instrument, the protections means being moveable to a position such that it can trap the sharp means for safe disposal.

A single unitary assembly maintains sterility and protects the user before use and provides a means to trap the sharp instrument after use. The protection assembly combines a cover to preserve sterility of a sharp instrument with a trap to prevent accidental sticks.

According to one embodiment the cover can be detached from the protection assembly and separately disposed. This embodiment has the further advantage that the separate disposal of the cover is cost saving as it is not contaminated. According to another embodiment the cover is permanently attached to the protection assembly. This embodiment has the further advantage that the assembly can be produced as one single mould (e.g. by means of injection moulding).

Instruments having a sharp means are any instrument or device having a sharp subject matter that must be maintained in a sterile condition prior to use, for example, syringe needles, infusion needles, acupuncture needles, phlebotomy needles, needles for medicament delivery devices (e.g. injection pens), catheters, trocars, cannulas, lancets, knives, scalpels and like instruments. Sharp means are parts of such instruments which are suitable to cause accidental pricking or cutting (e.g. scalpel- or lancet blades or the sharp parts or points of needles/cannulas etc.). One embodiment of the sharp means is formed as blade of e.g. a scalpel. An alternative embodiment of sharp means is formed as a needle, e.g. of a medication delivery device.

One embodiment of the cover is formed so that the sharp means is completely covered. An alternative embodiment of the cover covers only a part of the sharp means, e.g. needlepoint or blade. The cover prevents accidental pricking or cutting by the sharp means before use of instrument. Example materials for making the cover comprise, e.g. Polyethylentherephtalat (PET) or polyolefins (such as: polyesters—PE, polypropylenes—PP or polyvinyl chloride—PVC).

One preferred embodiment of the cover is configured to completely surround the sharp means of the instrument while maintaining sterility of the sharp means prior to use. The cover is attached to the sharp means and is subject to sterilization in any manner known to the art.

According to one embodiment, the cover has distal and proximal ends and a slit is located between the proximal and distal ends of the cover. The slit is initially covered by a thin membrane to preserve sterility of the sharp means before use. The slit is preferably located along a plane that makes it convenient to remove the cover in a sideways or horizontal direction relative to the axis of the instrument. The slit is configured and designed to allow the cover to be removed from the sharp means in sideways fashion as opposed to the traditional manner, which is pulling it off in an axial direction. The dimension or width of the slit depends on the materials of construction of the cover and the size and shape of the sharp means. The slit is covered with a thin pierceable membrane. This membrane covers or encloses the entire slit to maintain sterility until the instrument is ready for use.

The protection means prevents accidental pricking or cutting after use of the instrument. The user bends the protection or protections towards the sharp means so that it is trapped. The protection means has one or more trapping means, which may be integral part of the protection means, to cover at least a part of the sharp means.

One embodiment of trapping means is formed as shield having an opening. The protection means is moveable so that the sharp means is within the opening after use. An alternative embodiment of trapping means is formed to cover or wrap the sharp means. This embodiment may be formed as patch or foil of tough, imperforate, foldable material.

The protection means comprises a mount for attaching the protection means to the instrument. One embodiment of the mount is an elongated part of the protections means configured to be attached to the instrument by adhesive means or any other kind of connection, such as a mechanical connection, e.g. a snap connection. The mount may be integral part of the protection means. One embodiment of the mount comprises an adhesive label attachable to the instrument. The label may provide information about the instrument or a drug to be delivered by the instrument.

Example materials for making the protection means or parts thereof such as the trapping means and/or the mount of the protection means comprise, e.g. Polyethylentherephtalat (PET) or polyolefins (such as: polyesters—PE, polypropylenes—PP or polyvinyl chloride—PVC).

A preferred embodiment of the protection means is formed as flexible tab configured to be bent by the user to a position such that it can trap the sharp means for safe disposal. The term “tab” refers to an elongated embodiment of protection means having a distal part that is suitable to trap the sharp means. A proximal part of the tab serves as mount.

In one embodiment the protection means is configured to be bent out of the way of the sharp means and then to return to the position such that the protection means can trap the sharp means. The protection means is bent in order to not affect the usage of the sharp means, e.g. during injection.

One embodiment of the protection means comprises a hinge incorporated between the mount and the distal end of the protection means. Any configuration of the hinge will work that allows the protection means to be bent easily towards the sharp means. Preferably the hinge allows the protection means to be bent in the horizontal direction (the vertical direction being approximally co-axial with the longitudinal-axis of the sharp instrument, e.g. the syringe). The hinge may be made of moving component or flexible material. The hinge may be of the same or of different material as the mount and/or the distal part of the protection means. In one embodiment the hinge is designed as cut out which forms a predetermined bending point.

In one embodiment the protection means comprises a self adhesive film. The protection means can be folded about the sharp means, so that the sharp means is encased between folded sidewalls of the protection means, the side walls being held together.

In an alternative embodiment the protection means comprises one or more clamp means configured to trap the sharp means. One embodiment of clamp means is designed to snap over the sharp means and trap it. The snap means may be formed as hook.

According to one embodiment of the protection assembly, the cover and the protection means are removeably connected. Before use, the cover and the protections means are separated and the cover is removed from sharp means before use. Separation may be performed before removing the cover or during removing the cover. The cover and the protection means can e.g. be connected with a breakable bridge. The bridge may be formed as predetermined breaking point. One embodiment of the bridge is made of the same material as the cover and the protection means.

In an alternative embodiment of the protection assembly, the cover is permanently attached to the protection means. In this embodiment, the cover is not separated from the protection means before and during use.

In one embodiment, a tether is provided, the tether being configured to permanently connect the cover and the protection means. One end of a tether is connected to the cover and the other end of the tether is connected to the protection means. After removing the cover remains connected with the protection means, then the protection means is bent away in order to not affect the usage of the sharp means. One embodiment, the tether is formed as elongated, flexible band. The tether may be integrally formed with the cover and the protection means, and it may be made of the same material. In an alternative embodiment, the tether and/or the cover and/or the protection means may be made of different materials.

According to one embodiment, the unitary protection assembly is be made of separate parts that are assembled in any possible manner known in the art that is suitable for permanent or breakable attachment of the protection means and cap (e.g. bonding, gluing or mechanical connection, such as snap connection).

According to another embodiment the unitary protection means is made in one piece (e.g. by injection moulding), wherein the protection means and cap are formed of a same material or of different materials.

The cover and the protection means may be made of polyethylentherephtalat (PET) or polyolefins (polyesters—PE, polypropylenes—PP or polyvinyl chloride—PVC). Preferably the protection assembly is integrally formed of the same material. In an alternative embodiment, the cover and the protection means are made of different materials. The cover and parts of the protection means may be molded. Adhesive foil may form trapping means or the mount.

Further embodiments are subject-matters of the dependent claims.

### BRIEF DESCRIPTION OF THE FIGURES

The invention will become more apparent from the detail description of embodiment contained below and will be described with reference to the accompanying drawings.

FIG. 1 shows a side view of one embodiment of the invention wherein the cover is a needle cover designed to be removed from the protection means formed as a tab.

FIG. 2 shows the embodiment of FIG. 1 attached to a syringe.

FIG. 3 shows the embodiment of FIG. 1 where the needle cover is removed from the tab.

FIG. 4 shows the embodiment of FIG. 1 where after injection the tab is used as a needle trap.

FIG. 5 shows a side view of another embodiment of the invention where the cover is a needle cover designed to remain connected to the protection means (tab).

FIG. 6 shows the embodiment of FIG. 5 in side view of the needle cover illustrating the slit used to expose the needle prior to use.

FIG. 7 shows the embodiment of FIG. 5 in an end view illustrating the slit cover by the membrane that encloses the needle before use.

### DETAILED DESCRIPTION

Although the unitary protection assembly according to the invention is described as having applicability primarily to a disposable syringe and needle assembly, the invention can easily be adapted to any instrument having a sharp subject matter that must be maintained in a sterile condition prior to use, for example, syringe needles, infusion needles, acupuncture needles, needles for medicament delivery devices (e.g. injection pens), trocars, cannulas, lancets, knives, scalpels and like instruments. For the sake of simplicity of understanding the invention the various embodiments have been described based on a needle and syringe instrument.

As mentioned, the invention can be adapted to work with a number of different instruments having sharp means which may formed as a “sharp” distal portion that must be maintained in a sterile condition prior to use, however, for ease of understanding, specific embodiments are described, the embodiments being based on use with a syringe and needle device, particularly a disposable needle and syringe, pre-filled or empty. The numerous embodiments of the invention will become evident by reviewing the figures, even though the illustrations are not full-scale, but instead are schematic in nature. Particularly, the thicknesses of the needle cover and flexible tab are exaggerated for illustration and description purposes.

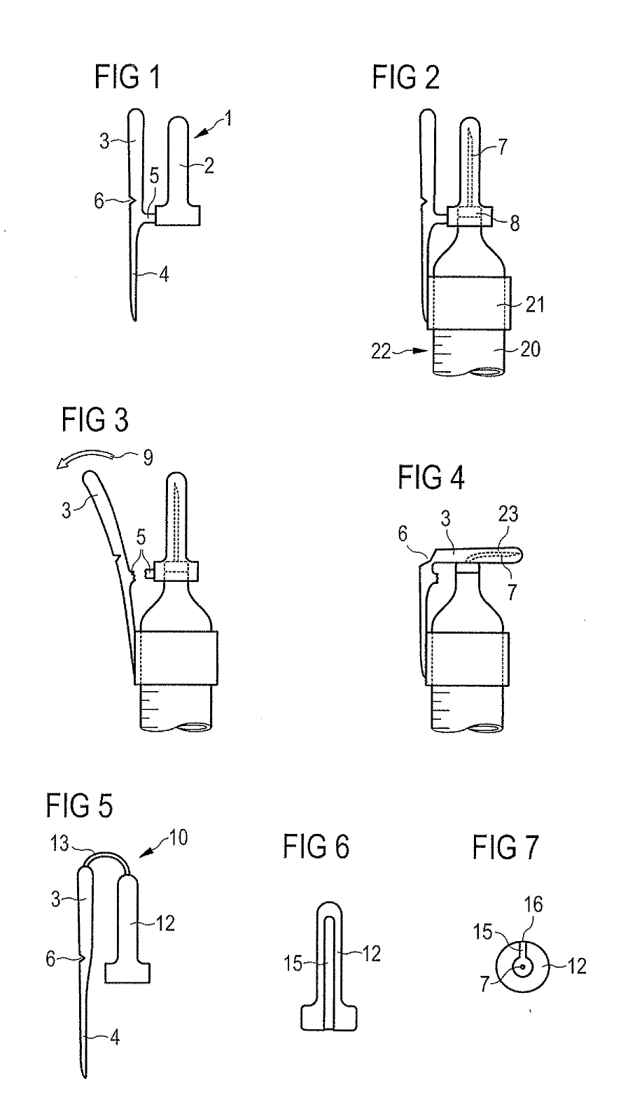

FIGS. 1 to 4 show one embodiment of the invention. There is provided a unitary needle protection assembly for a single use syringe having both needle sterility and anti-stick properties molded and manufactured as a single device for use with a syringe and needle. The assembly comprises, in combination, a needle cover having distal and proximal ends configured to completely surround a syringe needle while maintaining sterility of the needle prior to use; and a flexible tab removably connected to the needle cover, where the flexible tab has a syringe mount for attaching the tab with connected needle cover to a syringe barrel, wherein the flexible tab allows a user to separate the needle cover prior to injection, to move the tab away from an injection site during injection and then to return the tab to a position such that it can trap the needle for safe disposal.

Referring first to FIG. 1, there is shown one embodiment designated generally by reference number 1, where the protection assembly comprises a unitary design where a needle cover 2 is removably connected to flexible tab 3 serving as protection means through bridge element 5. Tab 3 has a mount 4 that is designed to attach to the body of an instrument containing a sharp distal end. In the case of a syringe, mount 4 is designed to be attached to the syringe barrel 20 (FIG. 2) by an adhesive, shrink wrapping, or any other attachment means known to the art. One preferred means is by incorporating mount 4 with, or as an integral part of, label 21 that is attached to syringe barrel 20 by physical or chemical means. Flexible tab 3, needle cover 2, and bridge 5 are manufactured as single unitary part using any material of construction provided that bridge 5 can be easily broken to allow needle cover 2 to be separated from flexible tab 3 as illustrated in FIG. 3. Preferably protection assembly 1 is formed using an inexpensive raw material such as a low grade polymeric material and fabricated using a high speed molding process. However, metal foils, paper/plastic laminates, and other flexible materials of construction could be used. An embodiment of the tab is to arrange it as a self adhesive film so that it can become directly attached to the syringe barrel. Particularly preferred is to use permanent adhesive adhesives, which are not water-soluble. One embodiment of the tab comprises clamp means suitable to trap the needle. Particularly suitable starting materials for the protection assembly include polyethylentherephtalat (PET) or polyolefins (polyesters—PE, polypropylenes—PP or polyvinyl chloride—PVC).

Needle cover 2 is fabricated such that is can completely cover and seal the sharp part of the instrument, illustrated in FIG. 2 as needle 7 attached to needle hub 8. Needle cover 2 is able to maintain the sterility of needle 7 prior to use. Preferably, for cost considerations and ease of manufacture, the needle cover should be made of the same material of construction as the flexible tab and bridge, however, it could be made of a different material.

Preferably, the protection assembly 1 is attached to syringe 22 by the syringe manufacture or the company that pre-fills the syringe with medicament. In either case the syringe 22 having the protection assembly attached and covering the needle 7 is then subject to sterilization in any manner known to the art. The entire assembly, as illustrated in FIG. 2, is then packaged for end use by a health care practitioner or directly by a patient. The needle cover 2 will maintain the sterility of the needle 7 until such time that the device is to be used. The user will then pull on flexible tab 3 in direction 9 (FIG. 3) causing bridge 5 to break or otherwise separate, thus freeing needle cover 2 from attachment to flexible tab 3. The needle cover is then removed and discarded. The user then continues to pull back the flexible tab in direction 9 in order to prevent the tab from interfering with the needle during injection. Mount 4 remains attached to the syringe barrel 20 and provides an anchor point or fulcrum to allow the flexible tab to be bent out of the way of the needle. Preferably, the material of construction of the tab allows for this bending, but not breaking, and dose not have sufficient memory such that it will immediately return the an upright position when the user releases the tab. Although FIGS. 1-3 show bridge 5 as a particular design, any configuration of the bridge can be employed provided that the user can easily break the bridge to free the needle cover. Preferably, for cost considerations and ease of manufacture, the bridge should be made of the same material of construction as the flexible tab and needle cover, however, it could be made of a different material.

Once the injection of medicament is complete, the user pushes flexible tab 3 needle in the opposite direction of direction 9 towards needle 7. Flexible tab 3 at its distal end contains a trap means 23 that is configured to accept and partially, or fully, enclose needle 7 as illustrated in FIG. 4. In this manner the user is protected from an accidental stick. Depending on the gauge of the needle, flexible tab 3 and trap means 23 can be pushed further past the vertical (or axis) of the syringe to a generally horizontal position as illustrated in FIG. 4. To assist in achieving this horizontal position, hinge 6 is incorporated between the distal and proximal ends of tab 3. Although hinge 6 is shown as a cut out of the tab 3, any configuration of the hinge will work that allows the tab to be bent easily in the horizontal direction. Bending the needle and tab 3 horizontally will further reduce the chance of needle sticks and also has the added benefit of rendering the syringe inoperable, thus preventing improper use. The syringe, needle and protection assembly can now be disposed of in an appropriate sharps container.

FIGS. 5 to 7 show another embodiment. A unitary needle protection assembly for a single use syringe having both needle sterility and anti-stick properties, comprising, in combination, a needle cover having distal and proximal ends configured to completely surround a syringe needle while maintaining sterility of the needle prior to use; a slit in the needle cover located between the proximal and distal ends, where the slit is initially covered by a thin membrane to preserve sterility of the needle before use; and a flexible tab connected to the needle cover and having a syringe mount for attaching the tab and needle cover to a syringe barrel, where the flexible tab allows the needle cover to be moved during injection. After injection the tab is returned to a position such that it can trap the needle for safe disposal.

Turning to FIGS. 5-7, there is shown the above mentioned embodiment 10 of the protection assembly. Here needle cover 12 is permanently attached to flexible tab 3 through tether 13. Tether 13, as opposed to bridge 5, is designed to securely fasten needle cover 12 to tab 3, yet allow for a very flexible connection. Needle cover 12, as shown in FIG. 6 has a slit 15 running generally from the proximal end to the distal end of the needle cover 12. The slit 15 is preferably located along a plane that makes it convenient to remove the needle cover 12 in a sideways or horizontal direction relative to the axis of the syringe. The slit 15 is configured and designed to allow the needle cover 12 to be removed from the needle 7 and needle hub in sideways fashion as opposed to the traditional manner, which is pulling it off in an axial direction. The dimension or width of the slit 15 depends on the materials of construction of the needle cover 12 and the size and shape of the needle hub 8. Preferably the slit 15 is configured so that it can be snapped on or off the needle hub 8. Because maintaining the sterility of the needle 7 is an important feature, the slit 15 is covered with a thin pierceable membrane 16, as shown in

FIG. 7. This membrane 16 covers or encloses the entire slit 15 to maintain sterility until the syringe is ready for use.

To use the embodiment illustrated in FIGS. 5-7, the user first pulls the needle cover 12 in a generally horizontal or sideways direction such that the needle hub tears through the membrane 16. Tether 13 keeps the needle cover 12 attached to flexible tab 3. As with the embodiment shown in FIGS. 1-4 described above, the tab 3 is then bent in direction 9 to allow the user to complete the injection without interference from the tab 3 or the attached needle cover 12. Once the injection is complete the tab 3 is bent to a position that the tap 3 traps the needle 7. Preferably, the user can take an additional step and can push the tab 3 and the needle 7 further to a generally horizontal position causing the needle 7 to bend, thus rendering the syringe inoperable for further use.

Alternatively the tab 3 is bent in the opposite direction after injection so that the user can replace the needle cover 12 by snapping it over the needle hub and needle 7. At this point, accidental needle sticks are prevented and the syringe/protection assembly can be safely discarded in an appropriate container. The user can take an additional step and can push the tab 3 and needle cover 12 further to a generally horizontal position causing the needle 7 to bend, thus rendering the syringe inoperable for further use. The materials of construction of this embodiment can be the same as those described above for the embodiment shown in FIGS. 1-4.

The foregoing description of the specific embodiments will so fully reveal the general nature of the invention that others can, by applying current knowledge, readily modify and/or adapt for various application such specific embodiments without departing from the generic concept, and therefore such adaptations and modifications are intended to be comprehended within the meaning and range of equivalents of the disclosed embodiments. It is to be understood that the phraseology or terminology herein is for the purpose of description and not of limitation.

The means, materials, and steps for carrying out various disclosed functions may take a variety of alternative forms without departing from the invention. Thus, the expressions “means to . . . ” and “means for . . . ”, or any method step language as may be found in the specification above or the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same function can be used; and it is intended that such expressions be given their broadest interpretation within the terms of the following claims.

1. Unitary protection assembly for an instrument having sharp means, comprising
a cover configured to cover the sharp means and
a protection means having a mount for attaching the protection means to the instrument, the protections means being moveable to a position such that it can trap the sharp means for safe disposal.

2. Protection assembly according to claim 1, wherein the cover and the protection means are removeably connected.

3. Protection assembly according to claim 1, wherein the cover and the protection means are connected with a breakable bridge.

4. Protection assembly according to claim 1, wherein the cover is permanently attached to the protection means.

5. Protection assembly according to claim 4, wherein one end of a tether is connected to the cover and the other end of the tether is connected to the protection means.

6. Protection assembly according to claim 1, wherein the cover is configured to completely surround the sharp means for the instrument while maintaining sterility of the sharp means prior to use.

7. Protection assembly according to claim 6, wherein the cover has distal and proximal ends, a slit is located between the proximal and distal ends of the cover, the slit is initially covered by a thin membrane to preserve sterility of the sharp means before use.

8. Protection assembly according to claim 1, wherein the protection means is formed as flexible tab.

9. Protection assembly according to claim 1, wherein the protection means is configured to be bent out of the way of the sharp means and then to return to the position such that the protection means can trap the sharp means.

10. Protection assembly according to claim 1, wherein the protection means comprises a hinge incorporated between the mount and the distal end of the protection means.

11. Protection assembly according to claim 1, wherein protection means comprises a self adhesive film.

12. Protection assembly according to claim 1, wherein the protection means comprises clamp means configured to trap the sharp means.

13. Protection assembly according to claim 1, wherein the mount comprises a label attachable to the instrument.

14. Protection assembly according to claim 1, wherein the cover and the protection means are made of a same material.

15. Protection assembly according to claim 1, wherein the protection assembly is integrally formed.
